(19) United States
(12) Patent Application Publication
(10) Pub. No.: US 2008/0274187 A1
(43) Pub. Date: Nov. 6, 2008

(54) Hard capsule composition and method of use

(75) Inventor: Karl Wei Cao, Richmond (CA)

Correspondence Address: Karl Wei Cao, 6271 Nicolle Place, Richmond, BC, V7C 4V7, CA

(73) Assignee: J.C. Bright M. Ltd.

(21) Appl. No.: 11/799,775

(22) Filed: May 2, 2007

(51) Int. Cl. A61K 47/36; A61K 47/38; A61K 9/00

(52) U.S. Cl. 424/484; 514/777; 514/778; 514/781; 514/782

(57) ABSTRACT

A hard shell capsule composition and method which is gelatin, BSE, plasticizer and preservative free, which is less sensitive to temperature, humidity and climate changes during manufacture and storage while remaining dimensionally and microbially stable.

### BACKGROUND

Hard capsules are distinguished by being produced as empty capsules, which are in two pieces, which fit together, and being filled and closed only after production. The hard capsules are in most cases produced from a single-phase aqueous solution in the so-called dip process.

On detailed examination of this processing, it is clear that the two parts of the capsule must be very mechanically stable, especially since the filling machines are very fast-running and dimensional changes would have very adverse effects on the filling process. Since the two molded parts are tightly fitted together after filling, it is necessary that the capsule material have adequate dimensional stability.

It is an object of the present invention to create a hard capsule, which is adequately and dimensionally stable.

To date, hard capsules for pharmaceutical and nutraceutical dosage forms have been mainly produced from gelatin. However, gelatin has some crucial disadvantages. Thus, gelatin is a material of animal origin and is thus not kosher. In addition, there is always a slight residual risk of BSE because gelatin from cattle is preferably used to produce them. There are increasingly more countries not allowing the import or export of gelatin hard capsules. As well, obtaining a suitable gelatin is very complicated and requires strict monitoring of the process. Nevertheless, differences between batches are large because of the animal origin, which is subject to certain variability. Gelatin is very microbially susceptible because it represents a good nutrient medium for microorganisms. Appropriate measures must therefore be taken during production and use of such packaging materials. The use of preservatives is frequently indispensable.

Since gelatin is basically a brittle material of low flexibility, it must be plasticized appropriately; for example, plasticizers must be added in the form of low molecular weight compounds. These plasticizers, which are necessary frequently migrate from the shell into the filling ingredients and cause changes there. The shell also loses plasticizers and becomes brittle and mechanically unstable during the course of storage due to this migration.

It is an object of the present invention to provide a gelatin free hard capsule, which in turn is BSE and preservative free as well as microbially and dimensionally stable.

Numerous substances lead to interactions with gelatin, such as, for example, aldehydes, polyphenols, reducing sugars, multiply charged cations, electrolytes, cationic or anionic polymers etc., with crosslinking frequently occurring and the capsule then disintegrating or dissolving only very slowly or not at all. Such changes are catastrophic for a nutritional supplement or drug product because efficacy is lost. Many drugs also lead to interactions with gelatin. In some cases during storage there is formation of drug degradation products with, for example, an aldehyde structure, which lead to crosslinking of the gelatin. Since gelatin has both acidic and basic groups, it is clear that reactions easily occur with other charged molecules. Gelatin can also be cleaved by enzymes, thereby creating contamination by enzymes or bacteria, which release enzymes that may drastically alter the properties of gelatin.

Because of these many disadvantages, there has been no lack of attempts to replace gelatin wholly or partly in hard capsules. Attempts have therefore been made to find synthetic polymers, which can be employed to produce hard capsules.

For example, polyvinyl alcohol has been described for this purpose. However, polyvinyl alcohol has a slow rate of dissolution, likewise requires additional plasticizers, which in turn may migrate and which, as already described above, may alter the properties of the filling, and it may moreover become extremely brittle as a consequence of internal crystallization. In particular, the flexibility decreases drastically during the course of storage if the ambient humidity is low.

Other attempts have been made to manufacture a non-gelatin hard capsule. For example, using a method such as polymers obtainable by free-radical polymerization of at least one vinyl ester in the presence of polyether-containing compounds and where appropriate one or more other copolymerizable monomers and subsequent at least partial hydrolysis of the ester functions in the original monomers with the provision that in the absence of another copolymerizable monomer, thus the polyether-containing compound must have a number average molecular weight of ltoreq 10,000.

The method described in the above is a traditional method of making a hard capsule using a synthetic base, however, it is very complicated compared to the process of the present invention. The present invention provides an alternative to these methods as described above and is very novel and unique in its simplicity and manufacture, thereby minimizing costly production and maintenance, which is beneficial to the environment as well as human health.

Yet another traditional method of manufacturing hard capsules is characterized by comprising the steps of dispersing a water-soluble cellulose derivative in hot water and cooling the dispersion to effect dissolution of the water-soluble cellulose derivative in the water, adding and dissolving a gelling agent in the water-soluble cellulose derivative solution to give a capsule-preparing solution, dipping a capsule-forming pin into the capsule-preparing solution at a predetermined temperature, then drawing out the pin and inducing gelation of the capsule-preparing solution adhering to the pin. A specific method that is used involves dissolution of the gelling agent and the gelling aid in purified water at approximately 70 degrees Centigrade. The water-soluble cellulose derivative is dispersed in the solution, following which the dispersion is cooled to from 50 to 52 degrees Centigrade. The pins are dipped into the resulting solution, and then drawn out to form capsules. If the temperature of the dipping solution falls outside the range of 50 to 52 degrees Centigrade, the jelly-like viscosity of the solution undergoes a subtle change that prevents good adherence of the solution to the capsule-forming pins during dipping and makes it difficult to obtain a uniform capsule film. The need for strict temperature control of the jelly at such a high temperature so as to obtain uniform capsules places a large burden on equipment and other resources. It is also troublesome to maintain tight control of the operation.

It is a surprising object of the present invention to provide a method of manufacture, which does not require strict temperature regulation and creates a more stable hard capsule, which lessens the burden on equipment, the environment and other resources.

Hard gelatin capsules are widely utilized in the field of pharmaceutical and nutraceutical preparations due to ease of preparation and administration. However, conventional hard gelatin capsules have a problem in that the capsule film loses flexibility and suffers cracking or chipping if the capsules are packed with a hygroscopic agent, such as a powder or granular material, since the moisture contained in the capsule film is absorbed by its contents, such as a nutritional supplement or drug, making hard gelatin capsules limited as to the types of drugs that may be contained in conventional hard gelatin capsules in order to prevent embrittlement of the capsule films caused by this moisture reduction.

It is an object of the present invention to provide a non-gelatin hard capsule, which is surprising and unique, as the free moisture is blown away from the capsules during manufacture, and lock in moisture in its molecular structure, thereby creating a more stable hard capsule.

It is an object of the present invention to increase the flexibility of hard capsules, which in turn is less sensitive to temperatures, climate and humidity related to manufacturing and storage conditions.

Also known in the art are hard film compositions for hard gelatin capsules having flexibility, which is increased by adding a plasticizer, such as glycerol, sorbitol, or polyethylene glycol to the gelatin. However, such hard gelatin capsules suffer problems in capsule manufacture in that the capsule films become too soft and the capsule drying speed is retarded due to the added plasticizer.

It is another surprising object of the present invention to provide a hard film composition for capsules, which will not be embrittled by a reduction in the moisture in the capsule film, even when a hygroscopic nutritional supplement or pharmaceutical drug is contained in the capsule. Further, the present invention eliminates the free moisture content, which minimizes the cracking, embrittlement and chipping of the capsules and thus prevents leakage and migration of the nutritional supplement or pharmaceutical drug contained within the capsules, which in turn also creates a more stable hard shell capsule that can be used with more types of pharmaceutical and nutraceutical preparations.

In addition, hard capsules for medical applications are frequently packed in so-called blister packs to increase the storage stability. When the hard capsules are pushed out of these packs, mechanical stress occurs and must not lead to deformation of the hard capsules. It is therefore necessary for hard capsules to have adequate mechanical stability.

### DETAILED DESCRIPTION OF THE INVENTION

Polysaccharides are a complex carbohydrate such as starch or cellulose, made up of sugar molecules linked into a branched or chain structure. The present invention is a polysaccharide composition, which becomes slightly thixotropic on forming a biphasic system containing casein micelles within a polysaccharide continuous network. When combined with xanthan gum, carrageenan and locust bean gum as it creates viscosity synergy as a framework with which the pullulan, water and sorbitol act as a filling agent, as well as eliminating the need for plasticizers, preservatives, magnesium chloride, or polymers or polyvinyl alcohol and the like.

Pullulan is a polysaccharide polymer consisting of maltotriose units, also known as -1,4-;-1,6-glucan. Three glucose units in maltotriose are connected by an -1,4 glycosidic bond, whereas consecutive maltotriose units are connected to each other by an -1,6 glycosidic bond. Pullulan is produced from starch by the fungus Aureobasidium pullulans. As an edible, mostly tasteless polymer, the chief commercial use of pullulan is in the manufacture of edible films that are used in various breath fresheners, gums and candy as well as oral hygiene products.

Xanthan Gum is a natural gum with a high molecular weight that is produced by the fermentation of glucose and is used in the food industry as a stabilizer. Xanthan gum is capable of synergistic interactions with galactomannans and glucomannans such as konjac mannan, E425; a non-ionic relatively rigid gelling, naturally partially acetylated, polysaccharide possessing a mixed (14)-linked -D-mannopyranose/-D-glucopyranose backbone with about 8%-(16)-glucosyl branchpoints. It synergistically forms thermoreversible soft elastic gels with locust bean gum on cooling mixtures; locust bean gum being preferred over guar gum as it has fewer galactose side chains and the interaction (here) concerning the smooth (14)-linked -D-mannopyranose backbone regions. A greater proportion of guar gum (80:20) is required for optimal synergy compared to locust bean gum (50:50) with the associating complex not requiring segments of unsubstituted backbone.

Locust bean gum is extracted from the endosperm of the seeds of the carob tree Ceretonia siliqua, which grows in Mediterranean countries. Locust bean gum is a galactomannana similar to guar gum consisting of a (14)-linked -D-mannopyranose backbone with branch points from their 6-positions linked to -D-galactose (i.e. 16-linked -D-galactopyranose). There are about 3.5 (2.8-4.9) mannose residues for every galactose residue. Locust bean gum is a polysaccharide made of the sugars galactose and mannose. In locust bean gum, the ratio of mannose to galactose is higher than in guar gum, giving it slightly different properties, and allowing the two gums to interact synergistically so that together they make a thicker gel than either one alone. Locust bean gum is commonly used in frozen preparations to prevent ice crystals from forming in the frozen preparation.

Carrageenans are naturally occurring linear sulphated polysaccharides, which fill the voids within the cellulose structure of certain species of Red seaweed known as Rhodophycae of the Solieriaceae, Gigartinaceae, Furcellariaceae, Phyllophoraceae, Hypneaceae, Rhabdoniaceae and Rhodophyllidaceae families. Carrageenan-yielding algae are grown in the Philippines, Indonesia, Canada, the USA, France, Korea, Spain, Portugal, Morocco, Mexico, Chile, Denmark and Brazil. Euchema cottonii and E. spinosum are main species producing k and i types which grow around the Philippines and Indonesia and other island coasts in the Far East; Chondrus crispus, a small cold-water seaweed producing k and l types is the most familiar red seaweed widely distributed around the coasts of the North Atlantic; the large cold-water Gigartina species from which k and l types are produced can be found from the cold deep coastal waters off Chile and Peru and Furcellaria species are collected in the cold waters around Northern Europe and Asia. The cold-water seaweeds are harvested once a year, whereas the warm-water seaweeds grow on a 3-month cycle. Carrageenan is generally considered a high-molecular-weight linear polysaccharide. Chemically, it comprises repeating galactose units and 3,6-anhydrogalactose (3,6-AG), both sulfated and non-sulfated, joined by alternating (1-)-and (1-4)-glycosidic linkages. The associated cations together with the conformation of the sugar units in the polymer chain determine the physical properties of the carrageenans. Carrageenans are used commercially as thickening, suspending, and gelling agents. Typical applications are as a thickener or binder in toothpaste, a suspending agent for cocoa in chocolate milk, and a gelling agent for milk puddings, water-gel deserts, and air-freshener gels.

Sorbitol is a sugar alcohol. Sorbitol is a white, sweetish, hygroscopic, crystalline sugar alcohol of six-carbon. It is found naturally in various berries and fruits or it is prepared synthetically by high-pressure catalytic hydrogenation of glucose sugar derived from cornstarch. It melts at 93 to 98 C depending on the form. It is used as a food additive, toothpaste, tobacco, toiletries and in cosmetics. It is used for vitamin-C fermentation. It is also used in the manufacture of polyethers for polyurethanes and surfactants. Sorbitol can be described as a glucose molecule with two hydrogens added. The two extra hydrogens are on either side of what used to be the double bond connecting the oxygen to the carbon, which is now a single bond.

### EXAMPLE 1

Mix Kappa-carrageenan in range percentages of 0.01 to 15.0 wt %; and xanthan gum in range percentages of 0.001 to 15.0 wt %, and water in range percentages of 10.0 to 50.0 wt % agitation at room temperature to 80 degrees Fahrenheit and allowed to create a biphasic system of long branched polysaccharide chains, much like a network or frame.

The aqueous solution consisting of water in range percentages of 60.0 to 90.0 wt % and sorbitol in range percentages of 0.001 to 15.0 wt % are blended into a liquid form at room temperature. The pullulan in percentage ranges of 20.01 to 30.0 wt % is then added as a dry powder to the aqueous solution at temperature ranges of room temperature to 80 degrees Fahrenheit, thereby creating a matrix solution. The pullulan solution with Potassium Acetate in range percentage of 0.03-0.1 wt %, is then added, which acts as a matrix solution or filling agent for the thixotropic solution, creating a thermally stable vehicle, which modifies any orifices or spaces, thereby reinforcing the integrity of the hard capsule. Once the solution is complete, heat to 70 degree Centigrade to completely dissolve components, vacuum to eliminate air bubbles and hold for use.

Hard capsules are then formed by dipping methods. After dipping, the dipping pins are subjected to raised temperature by blowing hot air around the hard capsules, and reduced humidity level in a dehumidified chamber, thereby causing the moisture content of the hard capsule to be blown away, thus forming a capsule which contains a constant amount of bonded moisture and is surprising and unique as the hard capsule is not temperature or humidity sensitive, brittle or migratory and are easy to produce with minimal effects to the environment.

The formulation based on the polysaccharide composition of example 1 of the present invention creates a hard capsule, which is adequately and dimensionally stable, gelatin free, preservative free, porcine free and BSE free as well as microbially stable, and does not use plasticizers in the manufacturing of hard shell capsules to maintain hard capsule flexibility. The formulation also increases the flexibility of hard capsules, which in turn makes the hard capsules resistant to sensitive temperatures and humidity related to use and storage conditions, embrittlement and migratory conditions when used with hygroscopic pharmaceutical and nutraceutical ingredients.

The formulation is adapted to inhibit microbial activity by reduction of free moisture from the hard capsules upon drying.

### EXAMPLE 2

Mix Kappa-carrageenan in range percentages of 0.001 to 15.0 wt %; and Sodium Alginate 0.005 to 15.0 wt % and water (10.0 to 50.0 wt %) are added together with agitation at room temperature to 80 degrees Fahrenheit and allowed to create a biphasic system.

The aqueous solution consisting of water (60.0 to 90.0 wt %) and manitol (0.001 to 15.0 wt %) are blended at room temperature. The pullulan (20.01 to 30.0 wt %) is then added as a dry powder to the aqueous solution at temperature ranges of room temperature to 80 degrees Fahrenheit. The pullulan solution with Potassium Acetate (0.03-0.1 WT %) and Sodium Lauryl Sulphate (0.0001-0.001% WT %), are then added. Once the solution mixing is complete, heat to 70 degree Centigrade to completely dissolve components, vacuum to eliminate air bubbles and hold for use.

Hard capsules are then formed by dipping methods. After dipping, the dipping pins are subjected to raised temperature by blowing hot air around the hard capsules, and reduced humidity level in a dehumidified chamber, thereby causing the moisture content of the hard capsule to be blown away, thus forming a capsule which contains constant amount of bonded moisture and is surprising and unique as the hard capsule is not temperature or humidity sensitive, brittle or migratory and are easy to produce with minimal effects to the environment.

### EXAMPLE 3

Mix Kappa-carrageenan in range percentages of 0.001 to 15.0 wt %; and Locust bean gum (0.005 to 15.0 wt % and water (10.0 to 50.0 wt %) are added together with agitation at room temperature to 80 degrees Fahrenheit and allowed to create a biphasic system.

The aqueous solution consisting of water (60.0 to 90.0 wt %) and sorbitol (0.001 to 15.0 wt %) are blended at room temperature. The pullulan (20.01 to 30.0 wt %) is then added as a dry powder to the aqueous solution at temperature ranges of room temperature to 80 degrees Fahrenheit. The pullulan solution with Potassium Acetate (0.03-0.1 wt %) and Ethylenediaminetetraacetic Acid (0.001-0.01% wt %), are then added, Once the solution mixing is complete, heat to 70 degree Centigrade to completely dissolve components, vacuum to eliminate air bubbles and hold for use.

Hard capsules are then formed by dipping methods. After dipping, the dipping pins are subjected to raised temperature by blowing hot air around the hard capsules, and reduced humidity level in a dehumidified chamber, thereby causing the moisture content of the hard capsule to be blown away, thus forming a capsule which contains constant amount of bonded moisture and is surprising and unique as the hard capsule is not temperature or humidity sensitive, brittle or migratory and are easy to produce with minimal effects to the environment.

### SUMMARY

A hard shell capsule composition and method which is gelatin, BSE, porcine, plasticizer and preservative free, which resists temperature, humidity and climate changes during manufacture and storage while remaining dimensionally and microbially stable.

What is claimed is:

1. A hard capsule composition comprising: a) from about 0.001 to 15.0 wt % carrageenan; b) from about 0.001 to 15.0 wt % locust bean gum; c) from about 0.001 to 15.0 wt % xanthan gum; d) from about 0.001 to 10.0 wt % sorbitol; e) from about 60.0 to 90.0 wt % water; f) from about 20.01 to 30.0 wt % pullulan; and g) from about 0.001 to 15.0 wt % sorbitol.

2. The composition of claim 1, wherein the polysaccharides are selected from the group consisting of alginates, agar gum, guar gum, locust bean gum (carob), carrageenan, tara gum, gum arabic, ghatti gum, Khaya grandifolia gum, tragacanth gum, karaya gum, arabian (araban), xanthan gum, gellan, starch, Konjac mannan, galactomannan, or funoran and the like.

3. The composition of claim 1, wherein the sugar alcohol is selected from the group sorbitol, mannitol and xylitol and the like.

4. The composition of claim 1, wherein the polysaccharide polymer is selected from the group cyanoethylpullulan, cyanoethylcellulose, acetylcellulose, cellulose, starch, pullulan and the like.

5. A hard capsule composition of claim 1, wherein the resulting biphasic polysaccharide phases are thixotropically networked subsequently while the aqueous matrix solution phase of the composition, which adheres and fills in the network frame of the composition.

6. A hard capsule composition of claim 5, wherein the resulting biphasic polysaccharide phases are thixotropically networked subsequently with the aqueous matrix solution phase of the composition, which adheres and fills in the network frame of the composition, thereby creating a hard capsule which is preservative free, and is microbially stable.

7. A hard capsule composition of claim 5, wherein the resulting biphasic polysaccharide phases are thixotropically networked subsequently with the aqueous matrix solution phase of the composition, which adheres and fills in the network frame of the composition, thereby creating a hard capsule, which resists temperature, humidity and climate changes during manufacture and storage while remaining dimensionally and microbially stable.

8. A hard capsule composition of claim 5, wherein the resulting biphasic polysaccharide phases are thixotropically networked subsequently with the aqueous matrix solution phase of the composition, which adheres and fills in the network frame of the composition, thereby creating a hard capsule which is gelatin, BSE, bovine and porcine free.

9. A hard capsule composition of claim 5, wherein the resulting biphasic polysaccharide phases are thixotropically networked subsequently with the aqueous matrix solution phase of the composition, which adheres and fills in the network frame of the composition, thereby creating a hard capsule which does not use plasticizers in the manufacturing of hard shell capsules to maintain hard capsule flexibility.

10. A hard capsule composition of claim 5, wherein the resulting biphasic polysaccharide phases are thixotropically networked subsequently with the aqueous matrix solution phase of the composition, which adheres and fills in the network frame of the composition, thereby creating a hard capsule which provides a method of manufacture, which does not require strict temperature regulation and creates a more stable hard capsule, which lessens the burden on equipment, the environment and other resources.

11. A hard capsule composition of claim 10, wherein the resulting biphasic polysaccharide phases are thixotropically networked subsequently with the aqueous matrix solution phase of the composition adheres and fills in the network frame of the composition, thereby creating a hard capsule which comprises 20.01 to 30.0 wt % pullulan before the method of blow drying to remove moisture from the hard capsules, thereby making the pullulan content of the finished hard capsule 85.01 to 90.0 wt %.

12. The hard capsule composition according to claim 1, further comprising coloring agents in a range from about 0% to 10% based upon the weight of the composition.

13. The hard capsule composition according to claim 12 wherein the coloring agent or mixture of coloring agents is selected from the group comprising consisting of azo-, quinophthalone-, triphenylmethane-, xanthene- or indigoid dyes, iron oxides or hydroxides, titanium dioxide or natural dyes.

14. The hard capsule composition according to claim 12 wherein the coloring agent or mixture of coloring agents is selected from the group consisting of carbon black, iron oxide black, iron oxide red, iron oxide yellow, titanium dioxide, riboflavin, carotenes, anthocyanines, turmeric, cochineal extract, clorophyllin, canthaxanthin, caramel, or betanin.

15. A method of claim 1, for manufacturing hard capsules comprising: a) from about 0.001 to 15.0 wt % carrageenan; b) from about 0.001 to 15.0 wt % locust bean gum; c) from about 0.001 to 15.0 wt % xanthan gum; d) from about 0.001 to 10.0 wt % sorbitol; e) from about 60.0 to 90.0 wt % water; f) from about 20.01 to 30.0 wt % pullulan; and g) from about 0.001 to 15.0 wt % sorbitol.

16. A method of claim 15, for manufacturing hard capsules which provides a method of manufacture, which does not require strict temperature regulation and creates a more stable hard capsule, which lessens the burden on manufacturing equipment, the environment and human resources.

17. A method of claim 15, for manufacturing hard capsules wherein the hard capsules blow dried before removing from the pins of conventional dipping process to remove moisture.

18. A method of claim 15, for manufacturing hard capsules, which is dimensionally stable.

19. A method of claim 15, for manufacturing hard capsules which comprises 20.01 to 30.0 wt % pullulan before the method of blow drying to remove moisture from the hard capsules, thereby making the pullulan content of the finished hard capsule 85.01 to 90.0 wt %.

20. A method of claim 15, for manufacturing hard capsules, which resists temperature, humidity and climate changes during manufacture and storage while remaining dimensionally and microbially stable.

21. A hard capsule shell of claim 1, which will not be embrittled by a reduction in the moisture in the capsule film, even when a hygroscopic nutritional supplement or pharmaceutical drug is contained in the capsule that can be used with more types of pharmaceutical and nutraceutical preparations.

22. A hard capsule shell of claim 1, which eliminates the free moisture content and minimizes the cracking, embrittlement and chipping of the capsules and thus prevents leakage and migration of the nutritional supplement or pharmaceutical drug contained within the capsules.

23. A hard capsule shell of claim 1, which eliminates the deformation of the hard capsules due to mechanical stress and has adequate mechanical stability.

24. A hard capsule shell according to claim 1, obtained by the dip process.
